(19) United States
(12) Patent Application Publication
(10) Pub. No.: US 2004/0115159 A1
(43) Pub. Date: Jun. 17, 2004

(54) Novel nanoemulsions

(76) Inventors: Charles C Tadlock, Islip Terrace, NY (US); Liliana George, Centerport, NY (US); Peter J Solis, Little Neck, NY (US); Raffi J Balian, Bay Shore, NY (US)

Correspondence Address: Karen A Lowney, Estee Lauder, 125 Pinelawn Road, Melville, NY, 11747, US

(21) Appl. No.: 10/471,044

(22) PCT Filed: Mar. 29, 2002
(86) PCT No.: PCT/US02/09630

(51) Int. Cl. A61K 7/075; A61K 7/08

(52) U.S. Cl. 424/70.22

(57) ABSTRACT

The invention provides oil-in-water nanoemulsions comprising as a primary surfactant a ternary system comprising at least one of each of an anionic surfactant, a bridging surfactant, and a cationic surfactant, preferably in a ratio of 1:≧1:≦1, with an overall substantially neutral charge. The nanoemulsions of the invention may be translucent or transparent, and provide a substantive and water-resistant vehicle for application of cosmetic, skin care or pharmaceutical products to skin and hair.

### FIELD OF THE INVENTION

The invention relates to cosmetic and pharmaceutical formulations. More specifically, the invention relates to nanoemulsions which may be transparent or translucent, and contain low levels of emulsifiers.

### BACKGROUND OF THE INVENTION

In recent years, the availability of clear personal care and cosmetic products has become increasingly important to the consumer. A clear product is perceived as light, clean, fresh and often cooling, features that can be important in certain types of products, for example, products for use in the sun, or makeup or skin care products to be used in warmer weather. The availability of such products, however, has frequently been limited to gels, soap-based sticks, and thin liquids. These forms, although obviously having their uses, are limited in the texture and feel that the final product can attain, and cannot approximate the classic cream or lotion feel conferred by standard oil-and-water emulsions. Achieving transparency, however, is not so easy when working from this type of base.

Transparent emulsions can be prepared in the form of microemulsions, in which the dispersed phase is essentially solubilized in the continuous phase by the action of a surfactant, and often, a cosurfactant. Rather than discrete isolated dispersed phase droplets, micelles comprising an inner core of the disperse phase material surrounded by a layer of surfactant are formed. These systems upon formation are normally clear, forming very small micellar droplets, but ordinarily require fairly large quantities of surfactant. Normally, it is desirable to keep surfactant levels low in cosmetic compositions, however, because they can strip the skin of its natural lipid barrier, and thus leave the skin more susceptible to irritants.

Alternate efforts to resolve this problem have turned to matching of refractive indices between the components of the two phases. Water and most water-soluble materials have a relatively low refractive index, i.e., about 1.3-1.4, whereas oils and oil soluble materials have refractive indices that range from about 1.4-1.5 for silicone oils, up to about 1.5-1.6 for hydrocarbons. In order to match the two phases, it is necessary to add a further component to the mix to either adjust the water phase index up, or reduce the oil phase index. Most frequently, the additional component is a glycol, usually in fairly high percentages, added to the water phase. Although the resulting product will appear to be clear, it is normally undesirable to use high levels of glycols in cosmetic products, since these compounds are themselves often irritating to the skin of the user. Additionally, since the amount of water that can stably be incorporated into a water-in-oil emulsion is limited, the use of large quantities of glycols in the aqueous phase necessarily reduces the amount of water, and the amount of water soluble actives, that can be used in the emulsion.

Currently, a frequently used vehicle for transparent products is a nanoemulsion. A nanoemulsion is defined as an oil-in-water emulsion in which the size of the dispersed oil droplets is less than 100 nm. Unlike microemulsions, these are true emulsions that ordinarily achieve the small sized oil droplets by processing the components under high pressure. Nanoemulsions provide an elegant, translucent or transparent vehicle that can be used for a variety of different product types. However, the nanoemulsion is a system in very delicate balance, subject to disruption if the wrong component is added, and not ordinarily capable of taking on too much oil. In addition, the known nanoemulsion systems may rely on the presence of relatively high levels of emulsifiers that are considered irritating, such as ethoxylated fatty ethers and esters. A truly desirable nanoemulsion should retain its transparency or translucency, be able to accommodate a reasonable amount of additives, such as fragrance or actives, while remaining stable, and utilize a low level of non-irritating emulsifiers, so as to remain mild and gentle on the skin of the user. The present invention provides such a nanoemulsion.

### SUMMARY OF THE INVENTION

The present invention relates to stable oil-in-water nanoemulsions comprising as its principle emulsifier a ternary system of surfactants comprising a cationic, anionic and bridging surfactant. The system is believed to provide a unique complex which, even when used at low levels, in combination with standard emulsion components, yields an attractive, translucent or transparent product that has a variety of possible uses. In a preferred embodiment, the components of the ternary system are used in a molar ratio of approximately 1:≧1:≦1 anionic: bridging: cationic, preferably with a range of up from about 1:1:0.1 up to about 1:3:1, and also preferably with an overall approximately neutral charge.

### DETAILED DESCRIPTION OF THE INVENTION

The oil-in-water nanoemulsions of the invention are prepared in much the same way as are other nanoemulsions. The aqueous phase may be any cosmetically acceptable water based material, such as deionized water, or a floral water. The oil phase may be any cosmetically or pharmaceutically acceptable oil, such an oil being defined for the present purpose as any pharmaceutically or cosmetically acceptable material which is substantially insoluble in water. As the oils can perform different functions in the composition, the specific choice is dependent on the purpose for which it is intended. The oils may be volatile or non-volatile, or a mixture of both. For example, suitable volatile oils include, but are not limited to, both cyclic and linear silicones, such as octamethylcyclotetrasiloxane and decamethylcyclopentasiloxane; or straight or branched chain hydrocarbons having from 8-20 carbon atoms, such as decane, dodecane, tridecane, tetradecane, and C8-20 isoparaffins.

Non-volatile oils include, but are not limited to, vegetable oils, such as coconut oil, jojoba oil, sunflower oil, palm oil, soybean oil; carboxylic acid esters such as isostearyl neopentanoate, cetyl octanoate, cetyl ricinoleate, octyl palmitate, dioctyl malate, coco-dicaprylate/caprate, decyl isostearate, myristyl myristate; animal oils such as lanolin and lanolin derivatives, tallow, mink oil or cholesterol; glyceryl esters, such as glyceryl stearate, glyceryl dioleate, glyceryl distearate, glyceryl linoleate, glyceryl myristate; non-volatile silicones, such as dimethicone, dimethiconol, dimethicone copolyol, phenyl trimethicone, methicone, simethicone; and nonvolatile hydrocarbons, such as isoparaffins, squalane, or petrolatum.

Water ordinarily will constitute from about 40-70% of the total emulsion, preferably about 50-65%, while the oil is ordinarily about 5-30% and preferably about 10-20% of the emulsion as a whole.

The ternary surfactant used is ordinarily added to the phase in which it is most compatible, using heat if necessary to accomplish better solubility or dispersibility, along with any active components which may be desired in the emulsion, and all components mixed together at low pressure.

The mixture is then subjected to high pressure mixing. By “high pressure” in the present context is meant a pressure of at least about 10,000 psi to about 20,000 psi; preferably about 11-17,000 psi. It is preferred that the emulsion is subjected to at least two passes, more preferably at least three passes under high pressure. There is no upper limit to the number of passes, but there appears to be no major improvement to be achieved after about seven passes. The preferred equipment is a microfluidizer, such as is available from Microfluidics. However, other suitable high pressure equipment is sold under the brand names Niro Soavi and Rannie. The particle size distribution is normally narrow, and very small, less than 100 nm, preferably with an average size of about 30-50 nm.

A crucial element of the nanoemulsion is the ternary surfactant system used to emulsify the nanoemulsion. The system comprises three essential elements, namely at least one anionic surfactant, at least one cationic surfactant and at least one bridging surfactant. Systems of this type are available commercially as premade blends from Stepan Company, Northfield Ill., identified as their C-A-N technology. The component surfactants can be added to the emulsion separately, but more often will be preblended before addition.

There is no restriction on the identity of the cationic or anionic surfactants. However, preferably, they are pharmaceutically or cosmetically acceptable surfactants. As a general rule, examples of such surfactants can be readily found, among other sources, in McCutcheon's Detergents and Emulsifiers, M.C. Publishing Company, North American Edition, 2000. The cationic surfactant can be selected from among fatty amine salts, fatty diamine salts, polyamine salts, quaternary ammonium salts, or polyoxyethylenated fatty amine salts, or combinations thereof, with anions being selected from, among others, halogen, sulfate, methosulfate, ethosulfate, tosylate, acetate, phosphate, nitrate, sulfonate, and carboxylate. In particular, the quaternary ammonium salts include mono-long chain alkyl-tri-short chain alkyl ammonium halides, wherein the long chain alkyl group has from about 8 to about 22 carbon atoms, and is derived from long chain fatty acids, and the short chain alkyl groups can be the same or different but preferably independently ethyl or methyl. Examples of useful quaternary surfactants include, but are not limited to, cetyl trimethyl ammonium chloride, lauryl trimethyl ammonium chloride, cocamidopropyl PG-dimonium chloride phosphate, cetrimonium bromide, cetrimonium chloride, hydroxycetyl hydroxyethyl dimonium chloride, quaternium 52, PPG-40 diethylmonium chloride, dicocodimonium chloride, behentrimonium chloride, quatemium-26, quatemium-60, isostearyl ethylimidazolinium ethosulfate, and dihydroxypropyl PEG-5 lineoleammonium chloride.

Useful salts of primary, secondary, and tertiary fatty amines include those having substituted or unsubstituted alkyl groups having from about 12 to about 22 carbon atoms. Suitable salts include the halogen, acetate, phosphate, nitrate, citrate, lactate, and alkyl sulfate salts. Amine salts derived from amine, such as stearamido propyl dimethyl amine, diethyl amino ethyl stearamide, dimethyl stearamine, dimethyl soyamine, soyamine, myristyl amine, tridecyl amine, ethyl stearylamine, N-tallow propane diamine, dihydroxy ethyl stearylamine, and arachidylbehenylamine, stearyl amine hydrogen chloride, soyamine chloride, stearylamine formate, N-tallow propane diamine dichloride and stearamidopropyl dimethylamine citrate. Other examples of useful quaternary ammonium compounds and amine salt compounds include imidazolines, imadazoliniums and pyridiniums, wherein the compound has at least one nonionic hydrophile containing radical, such as 2-heptadecyl-4.5-dihydro-1H-imidazol-1-ethanol, 4,5-dihydro-1-(2-hydroxyethyl)-2-isoheptadecyl-1-phenylmethylimidazolium chloride, and 1-[2-oxo-2] [[2-[(1-oxoctadecyl)oxy]ethyl]amino]ethyl]pyridinium chloride.

Anionic surfactants used in the present system are not limited in their nature, again however, preferably being pharmaceutically or cosmetically acceptable. The anionic surfactants include, but are not limited to, sulfates, such as linear and branched primary and secondary alkyl sulfates, alkyl ethoxysulfates, alkyl ether sulfates, fatty oleyl glycerol sulfates, alkyl phenol ethyoxylated sulfates, alkyl phenol ethylene oxide ether sulfates, C5-C17 acyl-N-(C1-C4 alkyl) and —N—(C1-C2 hydroxyalkyl)glucamine sulfates, and sulfates of alkylpolysaccharides such as the sulfates of alkyl polyglucoside; sulfonates such as salts of C5-C20 linear alkylbenzene sulfonates, alkyl ester sulfonates, C6-C22 primary or secondary alkane sulfonates, C6-C24 olefin sulfonates, alkyl glycerol sulfonates, fatty acyl glycerol sulfonates, fatty oleyl glycerol sulfonates, and C8-C18 alkyl sulfonates and alpha sulfonated C1-C6 alkyl esters of a fatty acid having an average of from about 11 to about 16 carbon atoms; carboxylates, such as alkyl ethoxy carboxylates, alkyl polyethoxy polycarboxylate surfactants, and their soaps, especially secondary soaps; a sulfosuccinate such as C8-22 sulfosuccinates; a sarcosinate, such as an alkali metal sarcosinate; or a sulfoacetate, such as C12-C20 alkyl sulfoacetates, for example, lauryl and myristyl sulfoacetate in the form of their sodium salts. A further useful anionic surfactant is the material known as surfactin, a naturally occurring product produced by fermentation of certain strains of Bacillus subtilis, and is commercially available from Showa Denko, KK, Japan. Preferred anionic surfactants, particularly for cosmetic purposes, are alkylaryl sulfonates, alkylaryl sulfonic acids, carboxylated alcohols, carboxylic acids, diphenyl sulfonate derivatives, ethoxylated fatty acids, fluorocarbon-based surfactants, isethionates, lignin and derivatives, olefin sulfonates, phosphate esters, phosphorus organic derivatives, polysaccharides, acrylic acids, and acrylamides, protein based surfactants, sarcosine, sulfates and sulfonics of oils and fatty esters, sulfates and sulfonics of ethoxylated alkylphenols, sulfates of alcohols, sulfates of ethoxylated alcohols, sulfosuccinates, and taurates. Particularly preferred for their mildness are glutamates, succinates, taurates, and isethionates.

The bridging surfactants used in the present invention include semi-polar nonionic surfactants, ethoxamide surfactants, and amphoteric surfactants. The presence of opposite polar or semi-polar charges on a surfactant molecule is key to the bridging requirement for the ternary system. Semi-polar nonionic surfactants include, but are not limited to, water soluble amine oxides, and water soluble sulfoxides, having at least one C10-18 alkyl moiety, and at least one moiety selected from the group consisting of alkyl groups and hydroxyalkyl groups containing from about 1-3 carbon atoms, or amine-oxide derived surfactants. Ethoxamides useful in the invention are ethoxylated alkanol amides or polyethylene glycol amides such as PEG-3 cocoamide or PEG-6 lauramide.

Preferably, the bridging surfactant is an amphoteric surfactant. Preferred are pharmaceutically or cosmetically acceptable amphoterics. Example of useful amphoterics include, but are not limited to, alkyl glycinates, propionates, imidazolines, amphoalkylsulfonates, N-alkylaminopropionic acids, N-alkyliminodipropionic acids, imidazoline carboxylates, N-alkybetaines, amido propyl betaines, sarcosinates, cocoamphocarboxyglycinates, amine oxides, sulfobetaines, or sultaines. Particularly preferred, especially for cosmetic use, are the betaine amphoteric surfactants, particularly amidopropyl betaines.

The choice of components for the system is preferably based on a total HLB for the system of about 5-12, and preferably an overall substantially neutral charge. The preferred combination of surfactants for cosmetic use is a glutamate as anionic surfactant, a quaternary ammonium salt as cationic surfactant, and an amidopropyl betaine as amphoteric. This combination is especially mild when applied to the skin and hair. It has been unexpectedly discovered that, although an equimolar ratio of surfactant components can be used, i.e., in a 1:1:1 ratio, for purposes of use in emulsifying a nanoemulsion, the ternary system is, in many embodiments, preferably used in a molar ratio of approximately 1 anionic: >1 bridging: 1 cationic. Alternately, however, when the bridging surfactant is a semi-polar nonionic surfactant, the proportion of cationic surfactant can be reduced to <1, in a range down to a level of about 0.1 up to less than 1, preferably in a range of from about 0.25 to about 0.5. Generally, the ternary combination, in the stated molar ratios, will be added to the composition in an amount ranging from about 0.5-5% total surfactant, preferably 1-3% total surfactant, by weight of the total composition. Ordinarily, the combination will be added as a preblend, but each component can also be added separately to the composition. Interestingly, it has also been found that, although each component is itself recognized as a surfactant, none of the components alone is adequate to achieve a stable nanoemulsion; all three must be used together.

Although the ternary system can be used alone, it is sometimes desirable to supplement the three main components with an additional small quantity of a secondary surfactant, preferably with an HLB of between about 3-5. Preferably, the secondary surfactant is a glyceryl fatty ester and derivatives thereof, such as glyceryl monostearate, glyceryl monolaurate, glyceryl monooleate, PEG 100 stearate, glycerol mono/distearate, polyglyceryl-2-stearate, fatty acid polyglycerol esters, hexaglycerol distearate, propylene glycol monolaurate and propylene glycol stearate. Other types of suitable secondary surfactants include hydroxylated lanolin, lanolin alcohol, ethoxylated castor oil, sorbitan monotallate, sorbitan monooleate, sorbitan sesquioleate, ethoxylated fatty amines, lecithin, oleth-2, palmitic/stearic acid esters, distilled monoglycerides, block copolymers of ethylene oxide and propylene oxide, cholesterol, dimethicone copolyol, and ethoxylated triglycerides, Normally, the secondary surfactant will be used in an amount of from about 0.5-5%, preferably about 0.5-2%, by weight of the total composition.

It also may be desirable to provide additional components, such as solubilizers, to the composition to enhance the clarity of the final nanoemulsion. Preferred for this purpose are alcohols, for example, short chain monohydric alcohols, such as ethanol or isopropanol, and/or polyhydric alcohols, such as butylene or propylene glycol, isopentyl diol, or amido-diols. The presence of a monohydric alcohol is strongly preferred, in an amount of from about greater than zero, to about 30%, preferably about 5-25%, and most preferred, at least about 10%, by weight of the composition. Polyhydric alcohols, if used, will be used in an amount of from about greater than zero to about 10%, preferably about 1-5%, by weight of the total composition.

Depending upon the intended use of the final product, it is also possible to provide the compositions with active agents or additives appropriate to the intended use. The preferred use of the compositions are as pharmaceutical or cosmetic vehicles, for application to the skin or hair. The nanoemulsions of the invention, in part because of the presence of the cationic surfactant, provide a very efficient delivery system for a wide variety of active components. The formulations are highly substantive to skin and hair; the emulsifying system exhibits a substantial degree of water resistance, and also potentially contributes to the preservation of the formulation as a whole. Examples of additional components that may be useful include, but are not limited to, agents for the eradication of age spots, keratoses and wrinkles, analgesics, anesthetics, anti-acne agents, antibacterials, antiyeast agents, antifungal agents, antiviral agents, antidandruff agents, antidermatitis agents, antipruritic agents, antiemetics, antimotion sickness agents, anti-inflammatory agents, antihyperkeratolytic agents, anti-dry skin agents, antiperspirants, antipsoriatic agents, antiseborrheic agents, hair conditioners and hair treatment agents, antiaging agents, antiwrinkle agents, antiasthmatic agents and bronchodilators, sunscreen agents, antihistamine agents, skin lightening agents, depigmenting agents, vitamins, corticosteroids, self-tanning agents, hormones, retinoids such as retinoic acid and retinol, topical cardiovascular agents, clotrimazole, ketoconazole, miconozole, griseofulvin, hydroxyzine, diphenhydramine, pramoxine, lidocaine, procaine, mepivacaine, monobenzone, erythromycin, tetracycline, clindamycin, meclocyline, hydroquinone, minocycline, naproxen, ibuprofen, theophylline, cromolyn, albuterol, topical steroids such as hydrocortisone, hydrocortisone 21-acetate, hydrocortisone 17-valerate, and hydrocortisone 17-butyrate, betamethasone valerate, betamethasone diproprionate, triamcinolone acetonide, fluocinonide, clobetasol, proprionate, benzoyl peroxide, crotamiton, propranolol, promethazine, vitamin A palmitate, vitamin E acetate and mixtures thereof.

It is also possible to add non-therapeutic or non-active agents, such as emollients, flavors, colorants, fragrances, gellants, thickeners, sunscreens, and the like, which enhance the ultimate use of the product, particularly for topical cosmetic or pharmaceutical purposes, and provided care is taken to avoid choosing components that will interfere with the clarity of the product. The final product can take the form of a milk, cream, lotion, gel, serum, or liquid spray, among others.

A particularly interesting use of the nanoemulsions of the invention is in the preparation of sunscreen formulations. It has been difficult to achieve a translucent product with the addition of sunscreens to other types of nanoemulsions. However, with the present nanoemulsion, the addition of sunscreen does not destroy the clarity of the product, thereby yielding a translucent sunscreen product which has been heretofore difficult to achieve. The nanoemulsions of the invention may therefore incorporate one or more sunscreen agents, such as benzophenones, avobenzone, cinnamates, salicylates, and the like.

The invention is further illustrated by the following non-limiting examples.

### EXAMPLES

### Example 1

A nanoemulsion of the following composition is prepared:

| Material | Wt. % |
|---|---|
| Phase I | |
| Purified water | 59.40 |
| Glycerin | 6.00 |
| Surfactant blend* | 5.50 |
| Isopentyldiol | 2.00 |
| Triethanolamine | 0.10 |
| Phase II | |
| Squalane | 5.50 |
| Cetyl alcohol | 0.50 |
| Ethanol | 15.00 |
| Phase III | |
| Cyclomethicone | 6.00 |

*contains: 19% sodium stearoyl glutamate, 7.5% cocamidopropyl PG-dimonium chloride phosphate, and 9% C16-C18 amidopropyl betaine, QS water to 100%.

The components above are combined as follows: The water phase components (Phase I) are heated to 65° C., and the oil phase components (Phase II) are heated to 75° C. When both phases have reached the proper temperature, the oil phase is added to the water phase under a Silverson homogenizer at about 5000-6000 rpm. This batch is then passed through a microfluidizer once, at a pressure of about 13,000-17,000 psi. The Phase III components are added under the Silverson homogenizer at about 5000-6000 rpm, and then the entire product is passed through a microfluidizer three more times. The resulting product is a translucent nanoemulsion which is stable for at least 4 weeks at 50° C.

Additional nanoemulsions are prepared using the following surfactant blends (all QS water to 100%):

(a) 20% sodium stearoyl glutamate, 8% cocamidopropyl PG-dimonium chloride phosphate, and 8% lauramidopropyl betaine

(b) 11.5% sodium stearoyl glutamate, 5% cocamidopropyl PG-dimonium chloride phosphate, 5% C16,18 amidopropyl betaine, 7% glyceryl monophosphate, 6% glyceryl stearate/PEG-100 stearate

(c) 9.5% sodium lauroyl sarcosinate, 5.5% cocamidopropyl PG-dimonium chloride phosphate, 7% C16,18 amidopropyl betaine, 7% glyceryl monostearate, 6% glyceryl stearate/PEG-100 stearate

(d) 19% sodium stearoyl glutamate, 8% cocamidopropyl PG-dimonium chloride phosphate, 9% C16,18 amidopropyl betaine, 12% glyceryl monostearate

(e) 13% sodium stearoyl glutamate, 5% cocamidopropyl PG-dimonium chloride phosphate, 6% C16,18 amidopropyl betaine, 8% glyceryl monostearate, 3% cyclodextrin

### Example 2

A sunscreen-containing nanoemulsion is prepared as follows:

| Material | Wt. % |
|---|---|
| Phase I | |
| Purified water | 53.90 |
| Glycerin | 6.00 |
| Triethanolamine | 0.10 |
| Surfactant blend* | 5.00 |
| Phase II | |
| Jojoba seed oil | 6.00 |
| Cetyl alcohol | 0.50 |
| Phase III | |
| Avobenzone | 1.50 |
| Octyl methoxycinnamate | 6.00 |
| Phase IV | |
| Ethanol | 15.00 |
| Phase V | |
| Cyclomethicone | 6.00 |

*Contains 24% isostearamidopropyl dimethylamine/isostearic acid, 20% sodium stearoyl glutamate, 10% glyceryl stearate, 10% glyceryl stearate, 3% stearic acid, QS water to 100%.

The formula described above is prepared as follows: The water phase and surfactant blend are heated to 65° C. The oil phase is heated to 75° C., and homogenized into the water. The mixture is passed through the microfluidizer once, and cooled to 30° C. Phases III and IV are added in sequence, then phase V is added. The resulting mixture is then passed through the microfluidizer as described in Example 1.

What we claim is:

1. An oil-in-water nanoemulsion comprising as primary surfactant a ternary surfactant system containing at least one of each of a cationic surfactant, an anionic surfactant, and a bridging surfactant.

2. The nanoemulsion of claim 1 in which the surfactants are provided in a molar ratio of approximately 1 anionic:≧1 bridging:≦1 cationic, and in which the system has an overall substantially neutral charge.

3. The nanoemulsion of claim 1 in which the cationic surfactant is selected from the group consisting of fatty amine salts, fatty diamine salts, polyamine salts, quaternary or diquaternary ammonium salts, polyoxyethylenated fatty amine salts, and combinations thereof.

4. The nanoemulsion of claim 1 in which the anionic surfactant is selected from the group consisting of glutamates, succinates, taurates, and isethionates.

5. The nanoemulsion of claim 1 in which the bridging surfactant is selected from the group consisting of include semi-polar nonionic surfactants, ethoxamide surfactants, and amphoteric surfactants.

6. The nanoemulsion of claim 5 in which the semi-polar nonionic surfactant is selected from the group consisting of water soluble amine oxides, water soluble sulfoxides having at least one C10-18 alkyl moiety, and at least one moiety selected from the group consisting of alkyl groups and hydroxyalkyl groups containing from about 1-3 carbon atoms, and amine-oxide derived surfactants.

7. The nanoemulsion of claim 5 in which the ethoxamide is selected from the group consisting of ethoxylated alkanol amides and polyethylene glycol amides.

8. The nanoemulsion of claim 5 in which the amphoteric surfactant is selected from the group consisting of alkyl glycinates, propionates, imidazolines, amphoalkylsulfonates, N-alkylaminopropionic acids, N-alkyliminodipropionic acids, imidazoline carboxylates, N-alkylbetaines, amido propyl betaines, sarcosinates, cocoamphocarboxyglycinates, amine oxides, sulfobetaines, and sultaines.

9. The nanoemulsion of claim 5 in which the surfactant is an amphoteric surfactant.

10. The nanoemulsion of claim 1 which comprises a glutamate as an anionic surfactant, an amidopropyl betaine as a bridging surfactant, and a quaternary or diquaternary ammonium salt as a cationic surfactant, in a molar ratio of approximately 1:≧:≦1

11. The nanoemulsion of claim 1 which also comprises a secondary surfactant selected from the group consisting of surfactants having an HLB of about 3-5.

12. The nanoemulsion of claim 11 in which the secondary surfactant is a glycerol fatty acid ester or derivative thereof.

13. The nanoemulsion of claim 1 which also comprises a solubilizer selected from the group consisting of monohydric alcohols, polyhydric alcohols, or a combination thereof.

14. The nanoemulsion of claim 13 which comprises a monohydric alcohol in an amount of at least about 10% by weight.

15. The nanoemulsion of claim 1 which also comprises a sunscreen.

16. An oil-in-water nanoemulsion comprising as primary surfactant a ternary surfactant system containing at least one of each of an anionic surfactant, a bridging surfactant and cationic surfactant in a ratio of about 1:≧1:≦1, wherein the anionic surfactant is selected from the group consisting of glutamates, succinates, taurates, isethionates, and combinations thereof; the bridging surfactant is an amphoteric surfactant selected from the group consisting of alkyl glycinates, propionates, imidazolines, amphoalkylsulfonates, N-alkylaminopropionic acids, N-alkyliminodipropionic acids, imidazoline carboxylates, N-alkylbetaines, amido propyl betaines, sarcosinates, cocoamphocarboxyglycinates, amine oxides, sulfobetaines, sultaines and combinations thereof; and the cationic surfactant is selected from the group consisting of fatty amine salts, fatty diamine salts, polyamine salts, quaternary or diquaternary ammonium salts, polyoxyethylenated fatty amine salts, and combinations thereof; wherein the ternary system has an overall substantially neutral charge.

17. The nanoemulsion of claim 16 in which the surfactants of the ternary system are present in a combined amount of from about 0.5 to about 5%.

18. The nanoemulsion of claim 16 which comprises a secondary surfactant having an HLB of from about 3-5, in an amount of from about 0.5% to about 5%.

19. The nanoemulsion of claim 18 in which the secondary surfactant is a glycerol fatty acid ester or derivative thereof.

20. The nanoemulsion of claim 16 which comprises a solubilizer selected from the group consisting of short chain monohydric alcohols, short chain polyhydric alcohols, amido-diols, and combinations thereof.

21. The nanoemulsion of claim 20 which comprises as solubilizer at least one short chain monohydric alcohol in an amount of at least about 10%.
